(19) United States
(12) Patent Application Publication
(10) Pub. No.: US 2024/0065687 A1
(43) Pub. Date: Feb. 29, 2024

(54) SUTURE ANCHOR WITH MULTIPLE LOAD ANGLES

(71) Applicant: Medos International Sari, Le Locle (CH)

(72) Inventors: David B. Spenciner, North Attleboro, MA (US); Gerome Miller, Randolph, MA (US)

(21) Appl. No.: 17/900,159

(22) Filed: Aug. 31, 2022

(51) Int. Cl. A61B 17/04

(52) U.S. Cl. CPC A61B 17/0401; A61B 2017/0412; A61B 2017/0464

(57) ABSTRACT

Suture anchors and methods for use are provided. A suture anchor can include an elongate body configured to be implanted in bone. The elongate body can have a distal and a proximal end, and at least one bone-engaging feature arranged on an outer surface. At least a portion of the elongate body can be attached to a soft tissue structure in need of being reattached to bone. The elongate body can have a delivery configuration that is different than a deployed configuration, and a change in the configuration of the elongate body can be effected by the application of at least two substantially opposed forces to the elongate body.

### FIELD

Suture anchors and methods are disclosed herein.

### BACKGROUND

Suture anchors can be used in orthopedic surgery to fix damaged and/or torn tissue during healing or other processes. In many instances, a patient may be suffering from multiple injuries simultaneously, and a suture anchor can be used to treat the multiple injuries. In a case of biceps tenodesis, for example, a biceps can be attached to the humerus in the bicipital groove using a suture anchor. There is a possibility that this same anchor could be used to reattach a torn rotator cuff tendon, such as the supraspinatus or subscapularis. In which case, the biceps tendon could be docked in the bicipital groove, and a stay suture could be used to tie down one part of a cuff tendon.

In such situations, however, the anchor is being used as a single point of fixation for two, nearly opposite force vectors, which could result in possible instability of the anchor.

Accordingly, there remains a need for improved suture anchors and methods of soft tissue repair in environments in which suture anchors are subjected to multiple force vectors.

### SUMMARY

In general, methods, systems, and devices for suture anchors are provided.

In an embodiment, a suture anchor is provided including an elongate body. The elongate body can be configured to be implanted into bone, and can have distal and proximal ends. The elongate body can include at least one bone-engaging feature arranged on an outer surface thereof. At least a portion of the elongate body can be configured to be attached to a soft tissue structure in need of being reattached to bone. The elongate body can have a delivery configuration that is different than a deployed configuration, and a change in the configuration of the elongate body can be effected by the application of at least two substantially opposed forces to the elongate body.

The suture anchor can vary in a number of ways and may include any of the following features, alone or in combination. For example, the elongate body can define an internal cavity configured to engage the soft tissue structure in the deployed configuration. For example, the elongate body can include at least one suture-engaging feature configured to engage a suture material to enable the soft tissue structure to be attached to the suture anchor via the suture material. For example, the elongate body can include a first portion and a second portion hinged to the first portion. In an aspect, the elongate body can define a cavity configured to receive and seat soft tissue, and application of the forces can cause the first portion and the second portion to pivot closer to each other. For example, the at least two substantially opposed forces can include a first force and a second force, and the first force and the second force can be offset from each other by about 100 to 180 degrees.

In an example, the elongate body can include first and second rotatable segments attached at a pivot. The first and second rotatable segments can have respective first and second proximal ends and respective first and second distal ends. The first and second proximal ends can be in closer proximity to each other in the delivery configuration than in the deployed configuration. The at least one bone-engaging feature can include a first bone-engaging feature protruding from the first distal end and a second bone-engaging feature protruding from the second distal end. The first and second distal ends can be in closer proximity to each other in the delivery configuration than in the deployed configuration. Tensioning the first rotatable segment in a first direction and tensioning the second rotatable segment in a second direction that is substantially opposed the first direction can cause the first and second rotatable segments to rotate relative to each other to transform the elongate body from the delivery configuration to the deployed configuration.

The suture anchor can further vary in a number of ways, and may include any of the following features, alone or in combination. For example, a dimension of the elongate body can increase when the elongate body is transformed from the delivery configuration to the deployed configuration. For example, the first rotatable segment can be configured to anchor a first suture at the proximal end thereof and the first rotatable segment can be configured to anchor a bone tunnel at the distal end thereof via the first bone engaging segment. In an aspect, the first suture anchor can be anchored to a soft tissue structure. For example, transformation of the elongate body from the delivery configuration to the deployed configuration can case the first and second rotatable segments to move from an orientation in which the first and second rotatable segments are substantially parallel to each other to an orientation in which the first and second rotatable segments are substantially perpendicular to each other. For example, the first and second rotatable segments can be rotatably coupled to a third segment. For example, the first direction and the second direction can be offset from each other by about 100 to 180 degrees.

In another example, the suture anchor can include first and second anchor portions rotatably coupled to the elongate body. The at least one bone-engaging feature can include a first bone engaging feature protruding from the first anchor portion and a second bone-engaging feature protruding from the second anchor portion. Tensioning the first anchor portion in a first direction can cause the first anchor portion to move in a second direction that is substantially opposed to the first direction, and tensioning the second anchor portion in the second direction can cause the second anchor portion to move in the first direction. Tensioning the first and second anchor portions can transform the elongate body between the delivery configuration and the deployed configuration.

The suture anchor can further vary in a number of ways, and may include any of the following features, alone or in combination. For example, a dimension of the elongate body can increase when the elongate body is transformed from the delivery configuration to the deployed configuration. For example, the first anchor portion can define a first aperture configured to receive a first tensioning suture, and the second anchor portion can defined a second aperture configured to receive a second tensioning suture. In an aspect, the first tensioning suture can be anchorable to a first soft tissue member and the second tensioning suture can be anchorable to a second soft tissue member. For example, the first bone-engaging feature can include a first barb extending in the first direction and the second bone-engaging feature can include a second barb extending in the second direction. For example, the elongate body can define a central cavity. The first and second anchor portions can each have proximal ends that are configured to extend into the central cavity. Within the central cavity, the first anchor portion can be configured to secure a first suture at the proximal end thereof and the second anchor portion can be configured to secure a second suture at the proximal end thereof. For example, the first direction and the second direction can be offset from each other by about 100 to 180 degrees.

A method of soft tissue repair is also provided. The method includes attaching two or more structures to a fixation element. The structures can include at least one of soft tissue structure to be reattached to bone and a flexible filament that is attached to soft tissue to be reattached to bone. The method also includes implanting the fixation element in bone. The method further includes applying a first tension to the fixation element disposed within bone via a first of the two or more structures. The method further includes applying a second tension to the fixation element disposed in bone via a second of the two or more structures. The second tension can be substantially opposed to the first tension. At least one of the first and second tensions can be applied to the at least one soft tissue structure.

### BRIEF DESCRIPTION OF THE DRAWINGS

This invention will be more fully understood from the following detailed description taken in conjunction with the accompanying drawings, in which:

FIG. 1A is a front view of an embodiment of a suture anchor;

FIG. 1B is a front view of the suture anchor of FIG. 1A in a delivery configuration;

FIG. 1C is a front view of the suture anchor of FIG. 1A in a deployed configuration;

FIG. 2A is a front view of another embodiment of a suture anchor;

FIG. 2B is a front view of the suture anchor of FIG. 2A in a delivery configuration;

FIG. 2C is a front view of the suture anchor of FIG. 2A in a deployed configuration;

FIG. 3A is a front view of another embodiment of a suture anchor;

FIG. 3B is a cross-sectional view of the suture anchor of FIG. 3A delivered into a bone tunnel;

FIG. 3C is a top view of the suture anchor of FIG. 3B in a delivery configuration;

FIG. 3D is a cross-sectional view of the suture anchor of FIG. 3B in a deployed configuration;

FIG. 3E is a top view of the suture anchor of FIG. 3B in a deployed configuration; and

FIG. 3F is a top view of the suture anchor of FIG. 3B in a deployed configuration, having distal and proximal tissue portions removed.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various systems, devices, and methods for anchoring soft tissue structures are provided. In some embodiments, the systems, devices, and methods allow for the anchoring of one or more tendons or soft tissue structures to a suture anchor affixed to bone. The one or more tendons or soft tissue structures can be attached to a suture anchor using forces applied in substantially opposite directions, and this application of forces can cause a transformation of the suture anchor to a configuration capable of harnessing the applied forces for increased anchoring within bone.

An exemplary suture anchor can have a delivery configuration in which the anchor is delivered to a bone tunnel. In a soft tissue repair procedure, such as to reattach soft tissue, the soft tissue can be attached to the suture anchor and the suture anchor can be anchored in bone by transitioning it from the delivery configuration to a deployed configuration as a result of the application of opposing tension vectors to the suture anchor. The opposing force vectors may result from forces applied to the suture anchor through the soft tissue that is reattached, and these forces help the suture anchor to more securely retain the suture anchor within the bone tunnel when the suture anchor is in the deployed configuration. The instruments described herein are merely intended to represent certain exemplary embodiments.

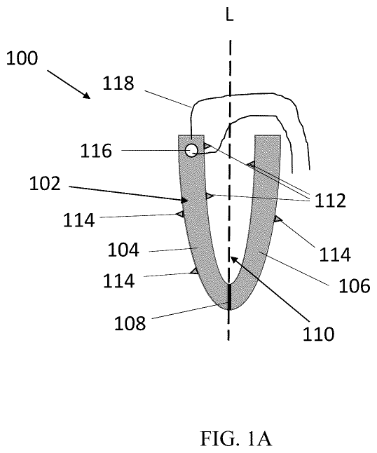

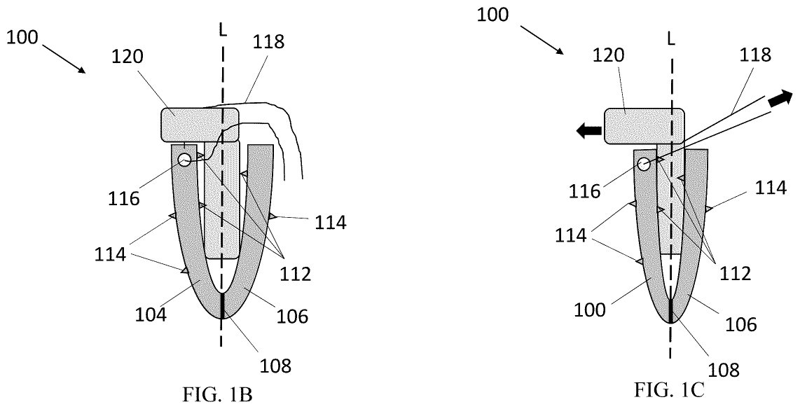

FIG. 1A depicts a suture anchor 100, according to an embodiment. The suture anchor 100 includes an elongate body 102 having a first portion 104 and a second portion 106. A middle region of the elongate body 102, where the first and second portions 104, 106 meet, includes a pivot point 108, which can take on various forms, e.g., a hinge. The elongate body 102 can fold about the pivot point 108, such that the first and second portions 104, 106 can move toward and away from each other, and the elongate body 102 takes on a substantially U-shaped configuration. In the U-shaped configuration, a central cavity 110 is defined between the first and second portions 104, 106. At least one tissue-engaging structure 112 can be disposed along the central cavity 110 on an internal face thereof. The tissue-engaging structure 112 can have one or more of a number of forms, e.g., a protrusion, a barb, a hook, a patterned area of increased frictional engagement, a combination thereof, etc. Opposite the central cavity 110, on an external face of the elongate body 102, can be at least one bone-engaging structure 114. The at least one bone-engaging structure 114 can be disposed on one or both of the first and second portions 104, 106, and the at least one bone-engaging structure 114 can have a same or a different form as the at least one tissue-engaging structure 112, i.e. a protrusion, a barb, a hood, a patterned area of increased frictional engagement, a combination thereof, etc. The suture anchor 100 can further include at least one suture-engaging structure 116 configured to secure a suture 118 thereto. The at least one suture-engaging structure 116 in FIG. 1A is shown in the form of a hole or an aperture, through which a suture 118 can be threaded. However, the suture-engaging structure 116 can vary in form to include a hook, ring, or other similar feature capable of engaging with the suture 118.

FIG. 1B depicts the suture anchor 100 in a delivery configuration. In the delivery configuration, the suture anchor 100 is pivoted about the pivot point 108 to be substantially-U shaped, and a soft tissue structure 120 is seated within the central cavity 110 (not shown). The soft tissue structure 120 can be any type of soft tissue (e.g., ligaments and tendons) that require repair or reattachment, and can include various tendons, such as, for example, a biceps tendon or a rotator cuff. The at least one tissue-engaging structure 112 within the central cavity 110 can grip the seated soft tissue structure 120 in order to keep the soft tissue structure 120 properly seated therein. When the suture anchor 100 is ready for deployment, the suture anchor 100 with a seated soft tissue structure 120 can be inserted into a prepared bone tunnel, pivot point 108 first. Once inserted, the at least one bone-engaging structure 114 can engage an interior of the bone tunnel to secure the suture anchor within the bone tunnel, thus making removal of the suture anchor 100 difficult or impossible. In addition, the act of inserting the suture anchor 100 into the bone tunnel can cause the first and second portions 104, 106 to pivot even closer together and clamp the seated soft tissue structure 120 even more, further assisting in keeping the soft tissue structure 120 seated in the central cavity 110 and securely reattached to bone.

After the suture anchor 100 is properly seated within the bone tunnel, the suture anchor 100 can be transformed into the deployed configuration through the application of substantially opposed force vectors, as schematically depicted in FIG. 1C. To transform the suture anchor 100, the suture 118 can be tensioned across the suture anchor 100 in a direction substantially opposed to the soft tissue structure 120, i.e., by forces that cross and/or are directed generally away from a longitudinal axis L of the suture anchor 100. This tensioning can cause the suture 118 to pivot the first and second portions 104, 106 even closer still, thereby further increasing the grip of the suture anchor 100 on the seated soft tissue structure 120. In this arrangement, tension is supplied to the suture anchor 100 by the suture 118 and by the soft tissue structure 120 in substantially opposed directions. For example, the angle between these tension vectors can be anywhere from approximately 100 to approximately 180 degrees.

In some variations, the first and second portions 104, 106 can have a gradient of stiffness, i.e., stiffer toward the hingepoint 108 and more flexible toward the at least one suture-engaging structure 116. In such variations, more flexible ends of the first and second portions 104, 106 could, under tension, grip the seated tissue structure 120, while the suture anchor 100 possessed a midpoint width similar to variations without having a gradient thickness in the first and second portions 104, 106. Maintaining a midpoint width could assist in retaining the suture anchor 100 within a bone tunnel, while the more flexible portions securely grip the seated tissue structure 120.

Although reference is made to orient the first portion 104 in a distal region and the second portion 106 in a proximal region, the reverse can be true as well, with the second portion in a distal region and the first portion 104 in a proximal region. Additionally, in some embodiments, the orientation may be such that there is no clear association with distal and proximal regions, and the suture anchor can be positioned to align about a separate orientation entirely. That is to say, the descriptions above are exemplary in nature and do not limit a precise delivery configuration of the suture anchor 100.

In certain embodiments, the tension supplied by the suture 118 and the tension supplied by the soft tissue structure 120 are directed to the device along an axis at least partially skew to the surface of a bone tunnel. This skewed application of forces can cause the suture anchor 100 to “toggle” or rotate slightly within the bone tunnel, such that the suture anchor 100 engages the bone tunnel at specific regions of the elongate body 102. In some embodiments, the at least one bone-engaging structure 114 can be located at one or more of these specific regions in order to assist in securing the suture anchor 100 within the bone tunnel.

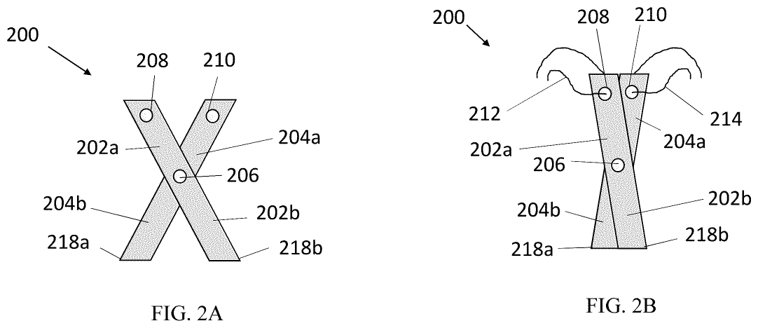

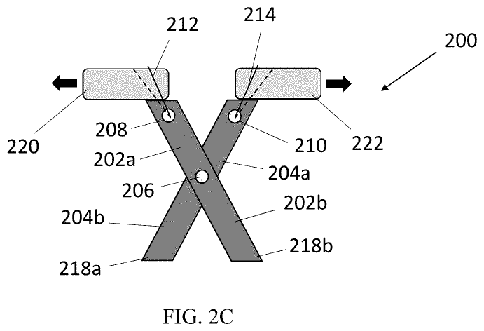

FIGS. 2A-2C depicts a suture anchor 200 according to another embodiment. Aside from the differences described in detail below, the suture anchor 200 is similar to suture anchor 100, and therefore common elements are not further described in detail.

The suture anchor 200 includes a first rotatable segment 202 having a first proximal end 202a and a first distal end 202b, and a second rotatable segment 204 having a second proximal end 204a and a second distal end 204b. The first and second rotatable segments 202, 204 are shown attached to a pivot point 206 at approximate middle portions thereof to form an “X” shape, as shown in FIG. 2A, however the first and second rotatable segments 202, 204 could be linked in any configuration, such as at end portions to form a “V” shape, at one middle portion and one end portion to form a “T” shape, or at portions elsewhere on the first and second rotatable segments 202, 204. The first and second rotatable segments 202, 204 can freely rotate about the pivot point 206 relative to each other.

The first and second proximal ends 202a, 204a include respective first and second suture-engaging structures 208, 210 in the form of holes or apertures, which can receive first and second sutures respectively. Similar to the suture-engaging structures 208, 210, the suture-engaging structures 208, 210 can take on other forms, such as a protrusion, a hook, a ring, etc. The first and second distal ends 202b, 204b respectively include at least one first and second bone-engaging structures 218a, 218b. The at least one bone-engaging structure 218 is shown in FIG. 2A as being a more-pointed region of the first and second rotatable segments 202, 204, however, the at least one bone-engaging structure can have other forms, including, e.g., a protrusion, a barb, a hook, a patterned area of increased frictional engagement, etc.

FIG. 2B depicts the suture anchor 200 in a delivery configuration. As shown, the first and second rotatable segments 202, 204 are rotated about the pivot point 206 such that they are substantially parallel to one another. At the first and second proximal ends 202a, 204a, affixed to the first and second suture-engaging structures 208, 210, are first and second sutures 212, 214. The first and second sutures 212, 214 can be respectively secured to first and second soft tissue structures 220, 222, as shown in FIG. 2C. Once the sutures 212, 214 are secure, the suture anchor 200 can be delivered to a prepared bone tunnel. After delivery, both the first and second sutures 212, 214, can be tensioned, causing the first and second rotatable segments 202, 204, to pivot relative to each other to become substantially perpendicular to one another, thereby transforming the suture anchor 200 into a deployed configuration.

The deployed configuration of the suture anchor 200 is shown in FIG. 2C. In this deployed configuration, the tension by the first and second sutures 212, 214 in substantially opposed directions results in engagement between the at least one bone-engaging structure 218 and a bone tunnel, which aids in securing the suture anchor 200 to the bone tunnel. The greater the tension, the greater the rotation by first and second rotatable segments 202, 204, and the greater the frictional engagement between the suture anchor 200 and the bone tunnel.

In some embodiments, the suture anchor 200 can be delivered to the bone tunnel prior to affixing one or more of the sutures 212, 214, to the soft tissue structures 220, 222. Once the suture anchor 200 has been fully inserted, the one or more sutures 212, 214 can be affixed and tensioning can occur to secure the suture anchor 200 within the bone tunnel.

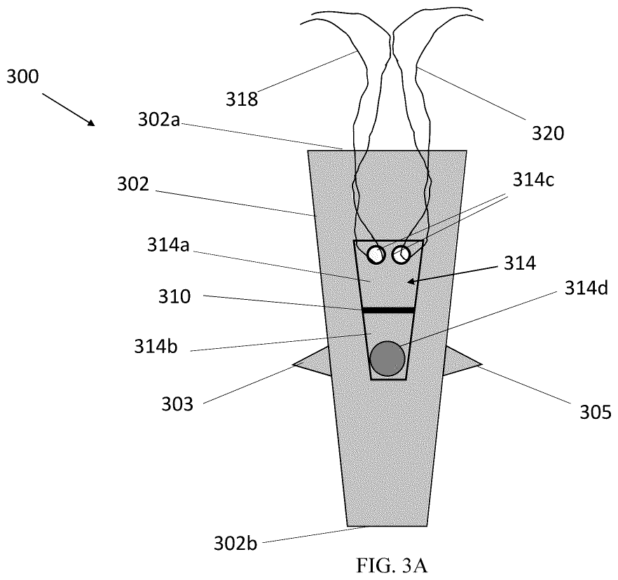

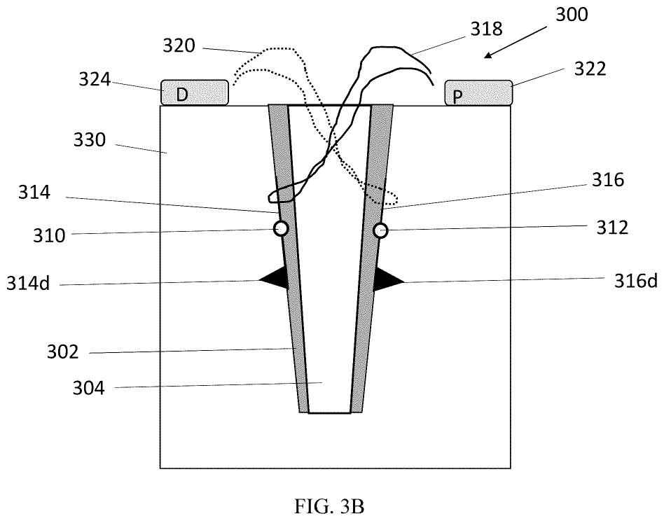

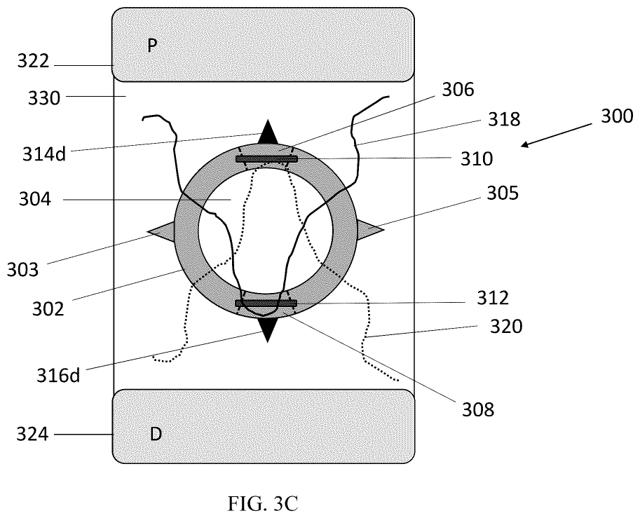

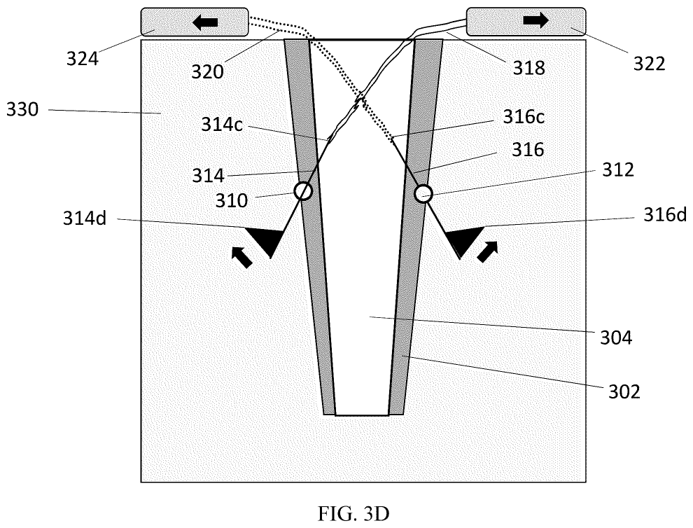

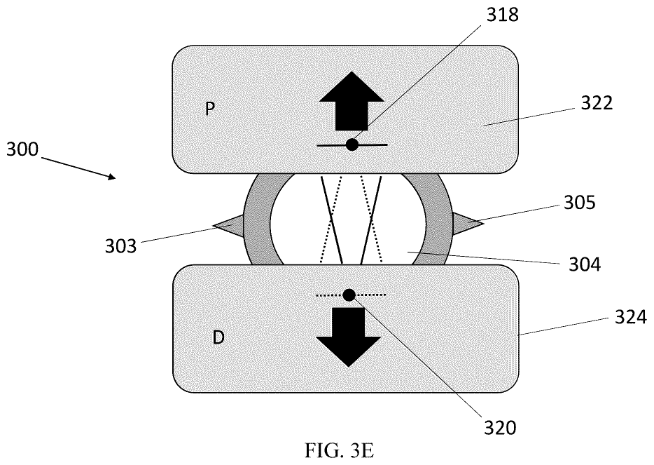

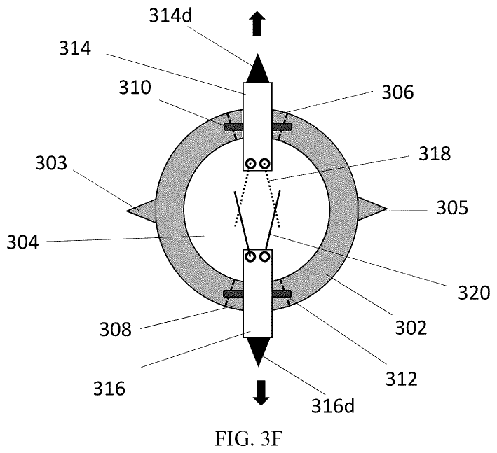

FIGS. 3A-3F, depict a suture anchor 300 according to another embodiment. Aside from the differences described in detail below, the suture anchor 300 is similar to suture anchors 100, 200, and therefore common elements are not further described in detail herein.

The suture anchor 300 can have a variety of forms and configurations, other than what is explicitly depicted. The suture anchor 300 includes an elongate body 302 having a slight taper from a top end 302a to a bottom end 302b thereof. The elongate body 302 defines a central cavity 304 (seen in FIGS. 3B-3F), tapered to match the taper of the elongate body 302, running through the entire length of the elongate body 302 from the top end 302a to the bottom end 302b. The elongate body 302 can include first and second apertures 306, 308 (seen in FIGS. 3C and 3F) disposed on opposed sides of the elongate body 302, e.g., a proximal side and a distal side. The first and second apertures 306, 308 are located approximately halfway up the elongate body 302, and they each are approximately the same size and shape. The first and second apertures 306, 308, are shown having a substantially trapezoidal appearance, as seen in FIG. 3A, however their exact form, location, and size can vary between embodiments, and even relative to each other. The elongate body 302 can further include at least one bone-engaging feature disposed on an outer surface thereof, and as seen in FIGS. 3A, 3C, 3E, and 3F, the at least one bone-engaging feature can be first and second bone-engaging features 303, 305 disposed on opposite sides of the elongate body 302.

Disposed within the first and second apertures 306, 308 are first and second pivot points 310, 312, each having respective first and second anchor portions 314, 316 rotatably coupled thereto. Although FIG. 3A depicts only the first anchor portion 314, the opposed side of the elongate body 302 includes the second anchor portion 316. The first and second anchor portions 314, 316, are substantially linear in shape and are coupled to first and second pivot points 310, 312 about respective middle portions thereof, and this coupling defines respective upper portions 314a, 316a— located above the first and second pivot points 310, 312— and respective lower portions 314b, 316b— located below the first and second pivot points 310, 312. Each of the upper portions 314a, 316a includes a suture-engaging feature capable of engaging and securing at least one suture thereto. As seen in FIG. 3A, a first suture-engaging feature 314c, located on the upper portion 314a of the first anchor portion 314, takes the form of a pair of holes, through which a first suture 318 is looped. While not explicitly shown, the second anchor portion 316 can have a comparable design and arrangement in order to engage and secure a second suture 320. As further seen in FIGS. 3A and 3B, a barb 314d, 316d protrudes from each of the lower portions 314b, 316b. These barbs 314d, 316d, are configured to engage a bone tunnel, and can take on a variety of forms as explained previously with respect to other bone-engaging structures.

During an implantation procedure, the suture anchor 300 begins in a delivery configuration, similar to what is depicted in FIG. 3A. The suture anchor 300 can be inserted into a bone tunnel 330, shown in FIG. 3B, up to a depth where the top end 302a of the suture anchor 300 is substantially flush with an outer surface of the bone tunnel 330. The suture anchor 300 can be oriented such that either of the first and second anchor portions 314, 316 is aligned in a proximal direction, while the other of the first and second anchor portions 314, 316 is aligned in a distal direction. This alignment can improve the effectiveness of the suture anchor 300 when engaging with soft tissue structures. As shown in FIGS. 3C-3E, the first anchor portion 314 can be aligned distally and the second anchor portion 316 can be aligned proximally. This alignment is presented by way of example, however, and the suture anchor 300 can be oriented in a number of ways, including with the first anchor portion 314 aligned proximally and the second anchor portion 316 aligned distally, or neither the first anchor portion 314 nor the second anchor portion 316 aligned proximally or distally.

First and second sutures 318, 320 can be pre-engaged by the first and second suture-engagement features 314c, 316c prior to insertion, or the first and second sutures 318, 320 can be engaged after insertion of the suture anchor 300 into the bone tunnel. In either scenario, the first and second sutures 318, 320 are threaded into the central cavity 304 and out of the suture anchor 300 through the top end 302a. From there, the sutures 318, 320 can be tensioned across the suture anchor in opposed directions, such that the barbs 314d, 316d flare outward from the elongate body 302 to engage the bone tunnel 330, i.e., if a suture 318, 320 is anchored to a proximal end of the suture anchor, the suture 318, 320 can be tensioned in a distal direction. This application of opposed tensions can occur, as described above, at an angle between 100 and 180 degrees.

The engagement of the barbs can be seen in FIGS. 3D-3F. FIG. 3D depicts a cross-sectional view of the suture anchor engaged in a bone tunnel 330, while FIGS. 3E and 3F depict a top-down view of the suture anchor 300 in a deployed configuration. The opposed tension vectors on the sutures 318, 320 cause the anchor portions 314, 316 to pivot first and second pivot points 310, 312 respectively. FIGS. 3D and 3E include first and second tissue structures 322, 324 connected to the first and second sutures 318, 320 respectively, while FIG. 3F omits the first and second tissue structures 322, 324. From the top-down perspective, the bone-engaging features 303, 305 can be seen disposed substantially orthogonal to a proximal direction and a distal direction. In some embodiments, the bone-engaging features 303, 305 can be disposed at other orientations and locations relative to the proximal and/or distal directions.

It will be appreciated that the terms “proximal” and “distal” are used herein with reference to a user, such as a clinician, gripping a handle of an instrument. Other spatial terms such as “front” and “rear” similarly correspond respectively to distal and proximal. It will be further appreciated that for convenience and clarity, spatial terms such as “vertical” and “horizontal” are used herein with respect to the drawings. However, surgical instruments are used in many orientations and positions, and these spatial terms are not intended to be limiting and absolute.

Values or ranges may be expressed herein as “about” and/or from/of “about” one particular value to another particular value. When such values or ranges are expressed, other embodiments disclosed include the specific value recited and/or from/of the one particular value to another particular value. Similarly, when values are expressed as approximations, by the use of antecedent “about,” it will be understood that here are a number of values disclosed therein, and that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value or within 2% of the recited value.

For purposes of describing and defining the present teachings, it is noted that unless indicated otherwise, the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety. Any patent, publication, or information, in whole or in part, that is said to be incorporated by reference herein is incorporated herein only to the extent that the incorporated material does not conflict with existing definitions, statements, or other disclosure material set forth in this document. As such the disclosure as explicitly set forth herein supersedes any conflicting material incorporated herein by reference.

What is claimed is:

1. A suture anchor, comprising:
an elongate body configured to be implanted in bone, the elongate body having a distal end and a proximal end, the elongate body including at least one bone-engaging feature arranged on an outer surface thereof,
wherein at least a portion of the elongate body is configured to be attached to a soft tissue structure in need of being reattached to bone,
wherein the elongate body has a delivery configuration that is different than a deployed configuration, and wherein a change in the configuration is effected by the application of at least two substantially opposed forces to the elongate body.

2. The suture anchor of claim 1, wherein the elongate body defines an internal cavity configured to engage the soft tissue structure in the deployed configuration.

3. The suture anchor of claim 1, wherein the elongate body includes at least one suture-engaging feature configured to engage a suture material to enable the soft tissue structure to be attached to the suture anchor via the suture material.

4. The suture anchor of claim 1, wherein the elongate body comprises a first portion and a second portion hinged to the first portion,
wherein the elongate body defines a cavity configured to receive and seat soft tissue, and
wherein application of the forces causes the first portion and the second portion to pivot closer to each other.

5. The suture anchor of claim 1, wherein the at least two substantially opposed forces include a first force and a second force, and wherein the first force and the second force are offset from each other by about 100 to 180 degrees.

6. The suture anchor of claim 1, wherein the elongate body comprises first and second rotatable segments attached at a pivot,
wherein the proximal ends of each of the first and second rotatable segment are in closer proximity to each other in the delivery configuration than in the deployed configuration,
wherein the at least one bone-engaging feature includes a first bone-engaging feature protruding from a distal end of the first rotatable segment and a second bone-engaging feature protruding from the second rotatable segment, the distal ends of each rotatable segment being in closer proximity to each other in the delivery configuration than in the deployed configuration, and
wherein tensioning the first rotatable segment in a first direction and tensioning the second rotatable segment in a second direction that is substantially opposed to the first direction causes the first and second rotatable segments to rotate relative to each other to transform the elongate body from the delivery configuration to the deployed configuration.

7. The suture anchor of claim 6, wherein a dimension of the elongate body increases when the elongate body is transformed from the delivery configuration to the deployed configuration.

8. The suture anchor of claim 6, wherein the first rotatable segment is configured to anchor a first suture at the proximal end thereof and the first rotatable segment is configured to anchor a bone tunnel at the distal end thereof via the first bone-engaging feature.

9. The suture anchor of claim 8, wherein the first suture is anchored to a soft tissue structure.

10. The suture anchor of claim 6, wherein transformation of the elongate body from the delivery configuration to the deployed configuration causes the first and second rotatable segments to move from an orientation in which the first and second rotatable segments are substantially parallel to each other to an orientation in which the first and second rotatable segments are substantially perpendicular to each other.

11. The suture anchor of claim 6, wherein the first and second rotatable segments are rotatably coupled to a third segment.

12. The suture anchor of claim 6, wherein the first direction and the second direction are offset from each other by about 100 to 180 degrees.

13. The suture anchor of claim 1, further comprising first and second anchor portions rotatably coupled to the elongate body,
wherein the at least one bone-engaging feature includes a first bone-engaging feature protruding from the first anchor portion and a second bon-engaging feature protruding from the second anchor portion, and
wherein tensioning the first anchor portion in a first direction causes the first anchor portion to move in a second direction that is substantially opposed to the first direction and tensioning the second anchor portion in the second direction causes the second anchor portion to move in the first direction, and wherein tensioning the first and second anchor portions transforms the elongate body between a delivery configuration and a deployed configuration.

14. The suture anchor of claim 13, wherein a dimension of the elongate body increases when the elongate body is transformed from the delivery configuration to the deployed configuration.

15. The suture anchor of claim 13, wherein the first anchor portion defines a first aperture configured to receive a first tensioning suture, and wherein the second anchor portion defines a second aperture configured to receive a second tensioning suture.

16. The suture anchor of claim 15, wherein the first tensioning suture is anchorable to a first soft tissue member and the second tensioning suture is anchorable to a second soft tissue member.

17. The suture anchor of claim 13, wherein the first bone-engaging feature comprises a first barb extending in the first direction and the second bone-engaging feature comprises a second barb extending in the second direction.

18. The suture anchor of claim 13, wherein the elongate body defines a central cavity,
wherein the first and second anchor portions each have proximal ends that are configured to extend into the central cavity, and
wherein, within the central cavity, the first anchor portion is configured to secure a first suture at a proximal end thereof and the second anchor portion is configured to secure a second suture at a proximal portion thereof.

19. The suture anchor of claim 13, wherein the first direction and the second direction are offset from each other by about 100 to 180 degrees.

20. A method of soft tissue repair, comprising:
attaching two or more structures to a fixation element, wherein the structures comprise at least one of soft tissue structure to be reattached to bone and a flexible filament that is attached to soft tissue to be reattached to bone;
implanting the fixation element in bone;
applying a first tension to the fixation element disposed within bone via a first of the two or more structures; and
applying a second tension to the fixation element disposed in bone via a second of the two or more structures, the second tension being substantially opposed to the first tension,
wherein at least one of the first and second tensions is applied to the at least one soft tissue structure.
